(19) United States
(12) Patent Application Publication
(10) Pub. No.: US 2024/0350399 A1
(43) Pub. Date: Oct. 24, 2024

(54) LONG-LASTING LUBRICATION COMPOSITION

(71) Applicant: LifeStyles Healthcare Pte. Ltd., Singapore (SG)

(72) Inventors: KC Nguyen, A. Phunphin, Surat Thani (TH); Atip Boombumrung, A. Phunphin, Surat Thani (TH); Chayapon Ngowprasert, A. Phunphin, Surat Thani (TH); Chayaporn Pongthanomsak, A. Phunphin, Surat Thani (TH); Yatima Kanghae, A. Phunphin, Surat Thani (TH); Andrea Pavlovicova, Brussels (BE)

(73) Assignee: LifeStyles Healthcare Pte. Ltd., Singapore (SG)

(21) Appl. No.: 18/578,064

(22) PCT Filed: Jul. 15, 2022
(86) PCT No.: PCT/SG2022/050501

§ 371 Date: Jan. 10, 2024

Related U.S. Application Data
(60) Provisional application No. 63/222,883, filed on Jul. 16, 2021.

(30) Foreign Application Priority Data
May 5, 2022 (AU) 2022901194

(51) Int. Cl. A61K 8/9789; A61F 6/04; A61F 6/08; A61F 6/14; A61H 19/00; A61Q 19/00

(52) U.S. Cl. CPC A61K 8/9789; A61F 6/04; A61F 6/08; A61F 6/14; A61H 19/00; A61Q 19/00; A61K 2800/34

(57) ABSTRACT

Disclosed herein are water-based lubricating compositions. The water-based lubricating composition may comprise at least one part or extract from an okra plant species and/or a flaxseed plant species. Also disclosed herein are articles comprising the water-based lubricating composition, possible applications and uses of the water-based lubricating composition, along with a possible method of synthesis.

### CROSS-REFERENCE TO RELATED APPLICATIONS

The present application claims priority from Australian Provisional Patent Application No 2022901194 filed on 5 May 2022, U.S. Provisional Patent Application No. 63/222,883 filed on 16 Jul. 2021, the contents of which are incorporated herein by reference in their entirety.

### TECHNICAL FIELD

The present disclosure relates to a water-based long-lasting lubricating composition. Disclosed herein is a personal water-based lubricating composition, comprising at least one part or extract from an okra plant species and/or a flaxseed plant species. In addition, also disclosed herein are articles comprising the water-based long-lasting lubricating composition, possible applications of the water-based long-lasting lubricating composition, and methods of synthesis.

### BACKGROUND

Silicone-based personal lubricants are well known for having lubricity and long-lasting properties as compared to water-based lubricants. However, one issue with a silicone-based lubricant is that it does not dry nor react directly with water, meaning regardless of whether it makes contact with sheets or a body part, the silicone-based lubricant may not be washed away easily. As such, silicone-based lubricants can cause stains, for example on bedding. There may also be difficulties in the removal of silicone-based lubricants. The lack of facile removal can require laborious cleaning, requiring the need for soap (to assist in solubilising the formulations), and/or the need for articles, such as bedding, to be laundered.

In addition, individuals prone to frequent yeast-infections may want to avoid using silicone-based lubricants, as these type of compositions tend to stay in the vagina until the silicone-based lubricant is expelled, which can take one or more days to occur.

Whilst water-based compositions could be used, there are known issues with these type of compositions, such as drying too fast when compared to silicone-based compositions, therefore providing insufficient lubricity, potentially causing, amongst other problems, discomfort for users of the water-based compositions.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

Previously water-based personal lubricating compositions may dry too fast following application to a surface, such as skin; especially when compared to a silicone-based lubricant. In addition, previously known water-based lubricating compositions may not provide long-lasting lubricity afforded by the silicone-based lubricant. One aim of the present disclosure is to provide a personal water-based lubricating composition which, which may possess one or more of the following characteristics: arguably easy to wash with water (for example from a body part or article); avoids or minimises staining (for example on a bed sheet); is based on or contains a majority of natural based ingredients; and/or provides long-lasting lubricity comparable to silicone-based lubricating compositions.

In addition, as a number of people prefer the silky texture and moisturising effect on skin provided by silicone-based lubricating compositions over water-based lubricating compositions, another possible aim is to provide a personal water-based lubricating composition that replicates these beneficial attributes of the silicone-based compositions, at least in part.

Silicone-based lubricants are Newtonian fluids where viscosity is independent of shear rate. As well as having low viscosity and low elastic contribution, silicone-based lubricants can flow without force applied and therefore have a tendency to drip out (for example leak), from tube packaging. On the other hand, the personal water-based lubricating composition described herein, in one embodiment, is preferably a non-Newtonian fluid and shows shear thinning behaviour in which viscosity decreases with increasing the shear rate and thus, its ability to easily spread on skin when applying (at high shear rate) and not dripping out (leak) from tube packaging (at very low shear rate). The flow behaviour of the personal water-based lubricant composition can be designed by tailoring, inter alia, the type and/or concentrations of one or more of the components, to minimise or mitigate at least the leaking issue.

In some embodiments, potential advantages of the disclosure herein, may include, but are not limited to one or more of:
the encompassed compositions are water-based (e.g. a reduced number of chemicals that may react with an individual's skin and cause irritation and/or an allergic reaction-low allergen)
the compositions comprise natural ingredients
similar to a comparative silicone-based personal lubricant, the compositions provide:
   a silky texture and/or
   a lubricity and long-lasting effect
the compositions comprise a high percentage of ingredients of natural origin (in some instances it may be possible that greater than about or equal to about 80%, for example greater than about or equal to about 95%, ingredients are of natural origin)
non-sticky
does not encourage and/or reduces the growth of moulds and/or yeasts
easy to wash off (especially in comparison to silicone-based or oil-based lubricants)
safe application and use with toys and/or a contraceptive device
non-hormonal
reduced, substantially no, or no residue following application to an article, surface and/or individual
reduced risk of staining an article when compared to a silicone-based or oil-based lubricant
the production of a non-Newtonian fluid and/or
minimised leakage, substantially no leakage, or no leakage from any packaging (for example a tube packing), containing a composition as described herein.

Additional benefits from the present disclosure can include one or more of:
formulation of a personal lubricant for penile and/or vaginal application
a composition that is compatible with natural rubber and/or polyisoprene articles, such as condoms
a composition that is pH balanced and paraben free and/or
a composition that is approved for gynaecological and/or dermatological applications.

In a first aspect, disclosed herein is a personal water-based lubricating composition comprising at least one of: a part or extract from an okra plant species; and a part or extract from a flaxseed plant species.

In a second aspect, disclosed herein is a massage gel or liquid comprising the personal water-based lubricating composition according to the first aspect.

In a third aspect, disclosed herein is a coating comprising the personal water-based lubricating composition according to the first aspect.

In a fourth aspect, disclosed herein is an article comprising the personal water-based lubricating composition according to the first aspect, or the coating of the third aspect.

In a fifth aspect, disclosed herein is use of the personal water-based lubricating composition according to the first aspect as a massage gel or liquid.

In a sixth aspect, disclosed herein is use of the personal water-based lubricating composition according to the first aspect as a coating.

In a seventh aspect, disclosed herein is use of the personal water-based lubricating composition according to the first aspect as a lubricant, wherein the lubricant composition is deposited directly on to at least a portion of skin on an individual.

It will be appreciated that the embodiments of each aspect of the present disclosure may equally be applied to each other aspect, mutatis mutandis.

### BRIEF DESCRIPTION OF DRAWINGS

Whilst it will be appreciated that a variety of embodiments disclosed herein may be utilised, described herein are a number of examples with reference to the following drawings:

FIG. 1—Flow curve of a silicone-based lubricant and water-based lubricants as defined herein.

FIG. 2—Coefficient of friction measurements for selected lubricants and water (average of 13 replicates).

FIG. 3—Image A) is a means comparison chart for the coefficient of friction for selected lubricants and water; and image B) is the interval plot for selected lubricants and water for measured coefficient of friction values.

FIG. 4—Retraction force for selected compositions.

FIG. 5—Image A) is a means comparison chart for determined axial forces (N) for selected lubricants and water; and image B) is the interval plot for selected lubricants and water for measured axial forces.

### DETAILED DESCRIPTION

### General Definitions and Terms

With regards to the definitions provided herein, unless stated otherwise, or implicit from context, the defined terms and phrases include the provided meanings. In addition, unless explicitly stated otherwise, or apparent from context, the terms and phrases below do not exclude the meaning that the term or phrase has acquired by a person skilled in the relevant art. The definitions are provided to aid in describing particular embodiments, and are not intended to limit the claimed invention, because the scope of the invention is limited only by the claims.

Herein “lubricating composition”, “lubrication composition”, “personal lubricating composition”, “composition”, “water-based lubricating composition” and “personal water-based lubricating composition” may be used interchangeably unless indicated otherwise or clear from context.

The recipients of the recited lubricating compositions are referred herein with the interchangeable terms “recipient” “individual”, and/or “subject”. These terms are used interchangeably and refer to a human being, of any gender, unless indicated otherwise or clear from context.

All publications discussed and/or referenced herein are incorporated herein in their entirety, unless described otherwise.

Unless otherwise required by context, singular terms shall include pluralities and plural terms shall include the singular. Throughout this disclosure, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the examples, steps, features, methods, compositions, formulations, and processes, referred to or indicated in this specification, individually or collectively, and any and all combinations or any two or more of said steps or features.

The term “and/or”, e.g. “X and/or Y” shall be understood to mean either “X and Y” or “X or Y” and shall be taken to provide explicit support for both meanings or for either meaning.

Unless otherwise indicated, the terms “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g. a “first” item) and/or a higher-numbered item (e.g. a “third” item).

As used herein, the phrase “at least one”, “one or more”, when used with a list of items, means different combinations of one or more of the listed items may be used and only one of the items in the list may be needed. The item may be a particular object, thing, or category. In other words, “at least one” or “one or more” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the disclosure can be presented in a range format. The range format is included for convenience and should not be interpreted as an inflexible limitation on the scope of the present disclosure. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 4.5 and 5, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term “consisting essentially of” is intended to exclude elements which would materially affect the properties of the claimed composition, method or process.

The terms “comprising”, “comprise” and “comprises” herein are intended to be optionally substitutable with the terms “consisting essentially of”, “consist essentially of”, “consists essentially of”, “consisting of”, “consist of” and “consists of”, respectively, in every instance.

Herein the term “about” encompasses a 10% tolerance in any value or values connected to the term.

Herein “weight %” may be abbreviated to as “wt %” or “wt. %”. The weight % may be w/w or w/v, unless specifically indicated or clear from context.

In some embodiments, “lubrication” may be regarded as the separation of a plurality of surfaces (wherein surfaces may include parts of one or more human bodies), otherwise in contact with each other, by a lubricant such that when the surfaces are in motion relative to each other the lubricant reduces frictional resistance at the interfaces associated with such motion, relative to the frictional resistance in the same system when the lubricant is absent. In some embodiments, lubrication utilising the compositions defined herein may prevent and/or lessen wear, irritation, heat generation, and/or damage to the surfaces due to such motion.

Herein personal lubricants include specialised lubricants (including massage gels or liquids), which serve to reduce friction with body tissues. Personal lubricants may be used to provide lubrication, or slip, during sexual activity. For example, personal lubricants can be used to increase pleasure and/or reduce pain during sexual intercourse, and can aid in reducing vaginal dryness. In medicine, personal lubricants may be employed for gynaecological examinations and the like. The personal lubricating compositions defined herein also encompass formulations that may be used as a massage gel or massage liquid.

Herein “water-based” means the lubricating composition has water as the main solvent and/or main ingredient.

Herein “natural ingredient” means an ingredient occurring in nature including from a natural origin. In a preferred embodiment, the natural ingredient is obtained from a renewable resource. Herein, a “natural ingredient”, for example a cosmetic ingredient, is from: a plant, an animal, a micro-organism or a mineral, including those obtained from these sources by:
physical processes (e.g. grinding, drying, distillation)
fermentation reactions occurring in nature and leading to molecules which occur in nature and
other procedures of preparation including traditional ones (e.g. extraction using solvents) without intentional chemical modification.
   Unless defined otherwise, the following materials, and materials originating from them, shall be considered to fall under the general heading of natural origin:
a) plants including fungi and algae
b) minerals
c) animals and
d) micro-organisms.
   Ingredients obtained from fossil fuels are excluded from the definition. In one or more embodiments, the natural ingredients described herein are plant-based or derived from a plant. In another embodiment, one or more natural ingredients comply with ISO 16128-2:2017.

Herein, the phrase “clear, substantially clear or translucent” is meant that the composition appears homogeneous, lacks visible precipitated solids, and is not opaque or cloudy. In addition, herein, “colourless,” means lacking perceptible or visually measurable colour or hue, that is, the substantial absence of all colour, which may be determined via appropriate means such as spectrophotometry.

Herein, “non-Newtonian composition” or “non-Newtonian fluid” refers to a composition or fluid whose viscosity is variable based on applied stress, i.e. whose viscosity changes when a gradient in flow speed changes.

Herein “non-sticky” means showing reduced or negligible adhesive or tacky characteristics, such that a composition, such as a lubricant applied to an article displays a reduced or negligible degree of cohesivity between portions of the article and/or a plurality of articles comprising the lubricant aggregating. Conversely, “sticky” as defined herein, means a composition, showing enhanced adhesivity or tack, such that the composition enhances aggregation of articles, or parts thereof.

### Lubricating Composition

### Components

Disclosed herein is a personal water-based lubricating composition, for example a personal water-based lubricating composition comprising at least one of a part or extract from an okra plant species; and a part or extract from a flaxseed plant species.

The water-based lubricating composition may in the form of a liquid or a gel. In one embodiment the water-based lubricating composition is in the form of a liquid. In one embodiment the water-based lubricating composition is in the form of a gel.

In one embodiment, at least one component of the lubricating composition is a natural ingredient. In another embodiment a plurality of components, for example two, three, four, five, or more, components of the lubricating composition are natural ingredients.

The natural ingredient may be an extract derived from a plant (including fungi and/or algae) or at least part of a plant. In one embodiment the plant extract is an extract obtained directly from a plant and not to a synthetic compound or composition. Unless defined otherwise, the term “plant” refers to multicellular living organism typically growing in a permanent site, absorbing water and inorganic substances through its roots and creating nutrients in its leaves by photosynthesis. Herein, unless defined otherwise, the term “plant extract” or “extract” refers to any extract obtainable from a plant or any portion thereof. The extract may be taken from any part of a plant such as the leaves, roots, flowers, seed, fruit, bark, or part thereof. The plant extract may be prepared from a single part of the plant, or from two or more parts of the plant. Herein, the “plant extract” or “extract” does not consist solely of water.

The natural ingredient may be used in any form. For example the natural ingredient in the form of a liquid, oil, gel, granule, a powder, or a mixture thereof, may be used in the formation of a lubricating composition. The natural ingredient may be used directly from the plant, or modified. For example the natural ingredient may be treated, for example dried to remove at least a portion of water (e.g. converted to a powder form). Herein, unless defined otherwise, a “gel” is a non-fluid colloidal network that is expanded throughout its whole volume by a fluid. In one embodiment, the natural ingredient is a gum.

A component in the form of a powder may be a “dry” powder, wherein the powder is substantially free of water. In one embodiment the powder has less than, or less than about: 10.0, 7.5, 5.0, 4.0, 3.0, 2.0, or 1.0% water by weight of the powder.

The natural ingredient may be mucilage obtained from a plant, or a part thereof. As used herein, “mucilage” is a thick, gluey (i.e. “sticky”) substance produced by a plant, comprising glycoproteins and exopolysaccharides.

A natural ingredient may be obtained by any process known in the art, or obtained commercially.

The lubricating composition may comprise one or more components selected from, but not limited to: natural gums, hemicelluloses (such as xyloglucans, xylans, and mannans), pectins (e.g. derived from citrus, apples, pears gooseberries, and the like), glycogen, chrysolaminarin, derivatives thereof, and mixtures thereof.

Herein “gum” or “natural gum” refers to a secretion of a plant, for example a tree or a shrub, which hardens on drying but is soluble or partly soluble in water.

Natural gums include non-cellulose polysaccharides derived from botanical or plant sources, seaweeds, or produced via bacterial fermentation. Non-limiting examples of plant-derived natural gums include, but is not limited to: sclerotium gel or gum, albizia gum, aloe mucilage, beta-glucan, chicle gum, dammar gum, fenugreek gum, glucomannan, guar gum, gum arabic (also called acacia gum), gum copal, gum ghatti, gum tragacanth, hakea gum, Hibiscus rosasinensis gum, honey locust gum, hupu gum, karaya gum, khaya gum, Lepidium sativum gum, locust bean gum, mastic gum, Mimosa scabrella gum, Mimosa pudica gum, okra gum, psyllium seed husks (also called ispaghula husk), spruce gum, Sterculia foetida gum, tamarind gum, tara gum, and derivatives of any of the foregoing. Examples of natural gums derived from seaweeds include, without limit, alginate or alginic acid, fucoidan, and laminarin derived from brown seaweeds, and agar and carrageenans derived from red seaweeds. Non-limiting examples of natural gums produced by bacterial fermentation include xanthan gum, gellan gum, dextran, welan gum, diutan gum, pullulan, and derivatives thereof.

Examples of plant extracts, optionally in the form of a gum, which may be present in the lubricating composition may be one or more compositions selected from, but not limited to: Abelmoschus esculentus (okra; e.g. as an extract or as a powder; CAS No. 84455-19-6 or CAS No. 91723 July 8); sclerotium gum (e.g. amigum, CAS No. 39464-87-4); xanthan gum (e.g. Keltrol® CG-T; CAS No. 11138-66-2); Limun Usitaissimum (e.g. flax, such as Limun Usitaissimum L gum; CAS No. 8001-26-1); Aloe (e.g. Glycine soja (soybean) oil, CAS No. 85507-69-3 and/or aloe barbadensis leaf extract, CAS No. 94349-62-8), and mixtures thereof.

A natural gum may be used in any form. For example the natural gum in the form of a liquid, oil, gel, granule, a powder, or a mixture thereof, may be used in the formation of a lubricating composition. The gum be used directly from the plant, or modified. For example the gum may be treated, for example dried to remove at least a portion of water, or diluted, for example diluted with water or a solvent comprising water.

In one embodiment, the lubricating composition comprises only: 1, 2, 3, 4, or 5 plant extracts. In one embodiment, the lubricating composition comprises at least: 1, 2, 3, 4, or 5 plant extracts.

In one embodiment, at least one plant extract (for example from okra and/or flaxseed), comprises mucilage. In another embodiment a plurality of plant extracts (for example from okra and/or flaxseed), comprise mucilage.

In one embodiment, at least one component of the lubricating composition is a natural gum. In another embodiment a plurality of components, for example two, three, four, five, or more, components of the lubricating composition are natural gums.

One or more components of the lubricating composition (such as part of an okra or flax part, and/or an extract of okra or flaxseed), may be present in the lubricating composition in an amount of about, or at least about: 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% w/w; or 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% w/v; or 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% v/v.

One or more components of the lubricating composition, for example a natural ingredient (e.g. a natural gum) may be present in the lubricating composition in an amount of about, or at least about: 0.5, 1, 2, 2.5, 5, 7.5, 10, 12.5, 15, 17.5, 20, 22.5, 25, 27.5, 30, 32.5, 35, 37.5 or 40% w/w; or 0.5, 1, 2, 2.5, 5, 7.5, 10, 12.5, 15, 17.5, 20, 22.5, 25, 27.5, 30, 32.5, 35, 37.5 or 40% w/v; or 0.5, 1, 2, 2.5, 5, 7.5, 10, 12.5, 15, 17.5, 20, 22.5, 25, 27.5, 30, 32.5, 35, 37.5 or 40% v/v.

In one embodiment, in the lubricating composition, non-water ingredients comprise or consist of about, or less than about: 60, 55, 50, 45, 40, 38, 36, 34, 32, 30, 28, 26, 24, 22, 20, 18, 16, 14, 12, 10, 8, 6, 4, or 2% w/w; or 60, 55, 50, 45, 40, 38, 36, 34, 32, 30, 28, 26, 24, 22, 20, 18, 16, 14, 12, 10, 8, 6, 4, or 2% w/v; or 60, 55, 50, 45, 40, 38, 36, 34, 32, 30, 28, 26, 24, 22, 20, 18, 16, 14, 12, 10, 8, 6, 4, or 2% v/v.

In one embodiment at least one component of the lubricating composition is an extract from at least one of: okra, flaxseed and/or aloe.

In one embodiment, at least one component of the lubricating composition is an part of an okra, flax and/or aloe plant, for example the leaves, roots, flowers, seed, fruit, bark, or part thereof; or an extract obtained from the: leaves, roots, flowers, seed, fruit, and/or bark of an okra, flax and/or aloe plant.

In one embodiment at least one component of the lubricating composition is from an okra plant species. In another embodiment the okra plant species belongs to the genus Abelmoschus. The okra plant species may be selected from, but not limited to: Abelmoschus esculentus (also sometimes referred to as Hibiscus esculentus), Abelmoschus caillei (also known as West African okra), Abelmoschus manihot, Abelmoschus ficuneus, Abelmoschus moschatus, or a mixture of any of the two or more species thereof. In various parts of the world, it is known as Okra, Ochro, Okoro, Quimgombo, Quingumbo, Ladies Fingers, Gombo, Kopi Arab, Kacang Bendi, Bhindi (S. Asia), Bendi (Malaysia), Bamia, Bamya or Bamich (middle east) or Gumbo (Southern USA). In one embodiment an extract is obtained from an okra fruit (pod) and/or okra seeds. In one embodiment the lubricant composition comprises a component of or derived from an okra plant species, which may be Abelmoschus esculentus, for example as a powder, liquid, oil, gel, granule, or a mixture thereof. In another embodiment the lubricant composition comprises: at least part of an okra seed or an extract thereof; at least part of an okra fruit (pod), or an extract thereof; or a mixture thereof.

In one embodiment the extract of okra (for example from an okra fruit (pods), and/or seed), comprises an okra mucilage.

In one embodiment at least one component of the lubricating composition is from flaxseed (also known as linseed or flax), which is the seed of a plant of the genus Linum in the family Linaceae. The term “flaxseed” includes the seeds of approximately 150 species of plant, all of which are within the scope of the invention. In one embodiment the flaxseed is from one or more species selected from: Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linurn perenne var. lewisii, Linum pratense and Linum trigynum, and mixtures thereof. In one embodiment, all varieties of all species are within the scope of the invention. Herein, the flaxseed or extract of flaxseed may be obtained from whole, broken, flaxseed flour and grinded and/or crushed flaxseeds. An extract of flaxseed may be a gum. Herein, an extract of flaxseed refers to a component and/or a part of the whole flaxseed. In one embodiment, an extract of flaxseed comprises mucilage. In another embodiment the extract of flaxseed may comprise oil obtained from flaxseed (or a portion thereof). In one embodiment the lubricant composition comprises a component of a flaxseed plant species or derived from an flaxseed plant species, which may be Linum usitatissimum, in a form such as a powder, liquid, oil, gel, granule, or a mixture thereof.

In one embodiment at least one component of the lubricating composition is from an Aloe plant species. The Aloe plant species may be selected from, but not limited to: Aloe vera, Aloe arborescens, Aloe aristata, Aloe dichotoma, Aloe nyeriensis, Aloe variegate, Aloe barbadensis, and Aloe wildii, or a mixture of any of the two or more species thereof. In one embodiment the lubricant composition comprises a component of or derived from an Aloe, for example as a powder, liquid, oil, gel, granule, or a mixture thereof.

Herein, a plant extract (for example an okra extract and/or a flaxseed extract), may comprise one or more of: a carbohydrate, a protein, dietary fibre (for example soluble and/or insoluble fibre), plant lignans, starch, sugar, fats, fatty acids (including for example at least one of: myristic acid, pentadecylic acid, margaric acid, stearic acid, nonadecylic acid, behenic acid, elaidic acid, vaccenic acid, linoelaidic acid, α-linolenic acid, palmitic acid, palmitoleic acid, olelic acid, linoleic acid, and mixtures thereof), phytosterols, and mixtures thereof. The plant extract may comprise one more fatty acids which is or are saturated, mono-unsaturated, di-unsaturated, tri-unsaturated, tetra-unsaturated, penta-unsaturated, hexa-unsaturated, or a mixture thereof.

Herein, a plant extract (for example an okra extract and/or a flaxseed extract), may comprise one or more amino acids selected from, but not limited to: alanine, arginine, aspartic acid, cysteine, glutamic acid, glycine, histidine, isoleucine, leucine, lysine, methionine, phenylalanine, proline, serine, threonine, tryptophan, tyrosine, valine, and mixtures thereof.

The plant extract (for example an okra extract and/or a flaxseed extract), may comprise omega-3 fatty acids, omega-6 fatty acids, or a mixture thereof. For example the plant extract may comprise at least one or more of: α-linolenic acid, eicosapentaenoic acid and/or docosahexanoic acid.

The plant extract (for example an okra extract and/or a flaxseed extract), may comprise one or more monosaccharides, polysaccharides, or a mixture thereof. The okra extract may comprise one or more of: mannose, rhamnose, glucuronic acid, glucose, fucose, arabinose, galacturonic acid, galactose, xylose, and mixtures thereof.

The plant extract (for example an okra extract and/or a flaxseed extract), may comprise protein and/or other minerals. Examples of minerals include, but are not limited to: calcium, potassium, phosphorous, selenium, sodium, magnesium, copper, iron, manganese, zinc, and mixtures thereof. The plant extract (for example an okra extract and/or a flaxseed extract), may comprise one or more vitamins, for example a vitamin selected from, but not limited to: vitamin A, vitamin B1, vitamin B2 vitamin D, vitamin E, and mixtures thereof. The plant extract (for example an okra extract and/or a flaxseed extract), may comprise arabinoxylans.

In one embodiment, an extract (for example an okra and/or flaxseed extract), may comprise non-water components, each independently in an amount of, about, or at least about: 0.1% w/w, 0.2% w/w, 0.3% w/w, 0.4% w/w, 0.5% w/w, 0.6% w/w, 0.7% w/w, 0.8% w/w, 0.9% w/w, 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, 15% w/w, 16% w/w, 17% w/w, 18% w/w, 19% w/w, 20% w/w, 25% w/w, or 30% w/w. In another embodiment, an extract (for example an okra and/or flaxseed extract), may comprise non-water components, each independently in an amount of, about, or at least about: 0.1% w/v. 0.2% w/v, 0.3% w/v, 0.4% w/v, 0.5% w/v, 0.6% w/v, 0.7% w/v, 0.8% w/v, 0.9% w/v, 1% w/v, 2% w/v, 3% w/v, 4% w/v, 5% w/v, 6% w/v, 7% w/v, 8% w/v, 9% w/v, 10% w/v, 11% w/v, 12% w/v, 13% w/v, 14% w/v, 15% w/v, 16% w/v, 17% w/v, 18% w/v, 19% w/v, 20% w/v, 25% w/v, or 30% w/v.

In one embodiment the total amount of non-water components in an extract (for example an okra and/or flaxseed extract), may be about, or at least about: 0.1% w/w, 0.2% w/w, 0.3% w/w, 0.4% w/w, 0.5% w/w, 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, 15% w/w, 16% w/w, 17% w/w, 18% w/w, 19% w/w, 20% w/w, 25% w/w, 30% w/w, 35% w/w, 40% w/w, 45% w/w, 50% w/w, 55% w/w, or 60% w/w. In another embodiment the total amount of non-water components in an extract (for example an okra and/or flaxseed extract), may be about, or at least about: 0.1% w/v, 0.2% w/v, 0.3% w/v, 0.4% w/v, 0.5% w/v, 1% w/v, 2% w/v, 3% w/v, 4% w/v, 5% w/v, 6% w/v, 7% w/v, 8% w/v, 9% w/v, 10% w/v, 11% w/v, 12% w/v, 13% w/v, 14% w/v, 15% w/v, 16% w/v, 17% w/v, 18% w/v, 19% w/v, 20% w/v, 25% w/v, 30% w/v, 35% w/v, 40% w/v, 45% w/v, 50% w/v, 55% w/v, or 60% w/v.

A component of the lubricating composition may have, or potentially have, a single function or multiple functions in the lubricating composition. For example a component may act as a humectant and/or a preservative, or as a humectant thickener.

One or more of the components of the lubricating composition may be an emollient, for example a natural ingredient is an emollient. In one embodiment provides the sensation of: moisturising, soothing and/or softening at least a portion of skin.

The lubricating composition may comprise at least one component which is: a humectant, emollient, antioxidant, preservative, thickener, perfume/fragrance, pH adjuster, pH buffer, emulsifier, surfactant, softener, moisturiser, cooling and/or tingling agent, or a mixture thereof.

Appropriate components may be identified by the person skilled in the art, with reference to appropriate materials, for example, applicable standards, and potentially websites such as CosmeticsInfo.org.

Examples of a humectant include, but are not limited to: 1,3-propanediol (CAS No. 504-63-2); hyaluronic acid or a salt thereof (for example sodium hyaluronate, CAS No. 9067-32-7), an extract of flax (e.g. Limun Usitaissimum L gum, CAS No. 8001-26-1), an okra extract (e.g. Abelmoschus Esculentus L. Moench, CAS No. 84455-19-6 or CAS No. 91723 July 8), an aloe vera extract (e.g. Glycine soja (soybean) oil, CAS No. 85507-69-3 and/or aloe barbadensis leaf extract, CAS No. 94349-62-8), and mixtures thereof.

One or humectants may be present in the lubricating composition in an amount (% w/w) of about, or at least about: 0.01, 0.02, 0.03, 0.04, 0.05, 0.06; 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0. % w/w.

Examples of preservatives include, but are not limited to: 1,3-propanediol (e.g. CAS No. 504-63-2), lactic acid (e.g. CAS No. 50-21-5), citric acid (e.g. CAS No. 77-29-9), tartaric acid (e.g. CAS No. 87-69-4), gluconic acid (e.g. CAS No. 526-95-4), potassium sorbate (e.g. CAS No. 24634-61-5), sorbic acid (e.g. CAS No. 110-44-1), sodium lactate (e.g. CAS No. 867-56-1), benzoic acid (e.g. CAS No. 65-85-0), and mixtures thereof.

One or preservatives may be present in the lubricating composition in an amount (% w/w) of about, or at least about: 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0% w/w.

Examples of thickeners include, but are not limited to: sclerotium gum (e.g. CAS No. 39464-87-4), hydroxy ethylcellulose (e.g. CAS No. 9005-62-0), xanthan gum (e.g. CAS No. 11138-66-2), an extract of flax (e.g. Limun Usitaissimum L gum, CAS No. 8001-26-1), an okra extract (e.g. Abelmoschus Esculentus L. Moench, CAS No. 84455-19-6 or CAS No. 91723 July 8), an aloe vera extract (e.g. Glycine soja (soybean) oil, CAS No. 85507-69-3 and/or aloe barbadensis leaf extract, CAS No. 94349-62-8), carboxypolymethylene polymers, and mixtures thereof.

One or more thickeners may be present in the lubricating composition in an amount (% w/w) of about, or at least about: 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0% w/w.

Examples of antioxidants include, but are not limited to benzoic acid (e.g. CAS No. 65-85-0).

### Water

The lubricating compositions disclosed herein are water-based. The water may be deionised water. The water may be referred to as “aqua”. The water may be purified water. In one embodiment, the water is pharmaceutical grade water, for example pharmaceutically acceptable water according to the British Pharmacopeia.

The water may be present in the lubricant composition in an amount of about, or at least about: 40, 42, 44, 46, 50, 52, 54, 56, 58, 60, 62, 64, 66, 68, 70, 72, 74, 76, 78, 80, 82, 84, 86, 88, 90, 92, 94, or 96% w/w; or 40, 42, 44, 46, 50, 52, 54, 56, 58, 60, 62, 64, 66, 68, 70, 72, 74, 76, 78, 80, 82, 84, 86, 88, 90, 92, 94, or 96% w/v; or 40, 42, 44, 46, 50, 52, 54, 56, 58, 60, 62, 64, 66, 68, 70, 72, 74, 76, 78, 80, 82, 84, 86, 88, 90, 92, 94, or 96% v/v.

### Natural Origin Calculation

In one embodiment at least one component of the lubricating composition is a natural ingredient. In another embodiment at least two, three, or four components of the lubricating composition are natural ingredients.

In one embodiment the selection of components for the lubricating composition is performed in accordance with ISO16128-2:2017 Cosmetics-Guidelines on technical definitions and criteria for natural and organic cosmetic ingredients. The contents of which are incorporated by reference.

In one embodiment the natural origin content percentage of the lubricating composition is calculated per ISO16128-2:2017. The contents of which are incorporated by reference.

Herein, unless defined otherwise “natural index” is as defined by section 4.3.1 in ISO16128-2:2017. The contents of which are incorporated by reference.

The natural index is a value indicating the extent to which a cosmetic ingredient meets the definition of natural ingredients from ISO 16128-1:2016, Clause 2. Unless defined otherwise, the value is assigned to each ingredient is according to the following guidance:
Natural index=1: Ingredient meets the definition of natural ingredients. The natural indexes of constitutive water, reconstitution water, extraction water and formulation water are each considered to equal 1.
Natural index=0: Ingredient does not meet the definition of natural ingredients.

The natural index of extract is calculated according to Formula (1):

$${I}_{n} = 1 - \frac{{m}_{DNS}}{{m}_{T}} \qquad ( 1 )$$

wherein:
Iₙ is the natural index;
m_{DNS} is the mass of derived natural solvent introduced; and
m_{T} is the total mass introduced (natural ingredients and ingredient solvents).
   For example, extracts of natural ingredients have a natural index of 1 if the solvents used are natural, including water.

In one embodiment at least one component of the lubricating composition has a natural index of 1. In another embodiment a plurality of components, for example 2, 3, 4, 5, or more, of the lubricating composition have a natural index of 1.

Herein, unless defined otherwise, the natural origin index is defined my section 4.3 in ISO16128-2:2017. The contents of which are incorporated by reference.

The natural origin index is a value indicating the extent to which a cosmetic ingredient meets the definitions of either natural ingredients in ISO 16128-1:2016, Clause 2, derived natural ingredients from ISO 16128-1:2016, Clause 3, or derived mineral ingredients from ISO 16128-1:2016, Clause 4.

The value is assigned to each ingredient according to the following guidance:
Natural origin index=1: Ingredient meets the definition of natural ingredients, constitutive water, reconstitution water, extraction water or formulation water. Extracts of natural ingredients using ingredient solvents that are natural or derived natural of wholly natural origin (according to ISO 16128-1:2016, Table A.1) have a natural origin index of 1.
0.5<Natural origin index≤1: Ingredient meets the definition of derived natural ingredients or derived mineral ingredients. The value is calculated as the ratio of the natural origin moiety, as determined by molecular mass, renewable carbon content or any other relevant methods, to the total molecular composition of that ingredient.
Natural origin index=0: Ingredient neither meets the definition of natural ingredients nor derived natural ingredients nor derived mineral ingredients, including those with natural origin indexes calculated to be ≤0.5.

In one embodiment at least one component of the lubricating composition has a natural origin index of 1. In another embodiment a plurality of components, for example 2, 3, 4, 5, or more, of the lubricating composition have a natural origin index of 1.

### Natural Origin Content.

In one embodiment the natural origin content of the lubricating composition is about or at least about: 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%. In another embodiment, the natural origin content of the lubricating composition is in the range of about 95 to about 100%. In yet another embodiment the natural origin content of the lubricating composition is greater than 99%.

In one embodiment, the lubricating composition does not comprise a silicon or silicone based compound.

### Grading of Components

A component and/or excipient of a lubricating composition described herein may be: food grade (i.e. safe for food use), cosmetic grade, United States pharmacopeia (USP) grade, British pharmacopeia (BP) grade European pharmacopeia (Ph. Eur) grade, national formulary (NF) grade, Japanese pharmacopeia (JP) grade, and/or Japanese pharmaceutical excipient (JPE) grade.

### Attributes

In one embodiment the lubricant composition is clear, substantially clear or translucent.

In one embodiment the lubricant composition is colourless, or substantially colourless.

In another embodiment, the lubricant composition possesses at least one of the following attributes: transparent or substantially transparent; free or substantially free of foreign matter; free or substantially free of any contaminating or potentially contaminating substances.

In one embodiment the lubricant composition is viable for gynaecological and/or dermatological applications. For example the lubricant composition may comply with the internationally recognised guidelines of the International Contact Dermatitis Research Group.

In one or more embodiments, the lubricating composition may be formulated to be odourless, or substantially odourless, and/or tasteless or substantially tasteless. An unflavoured and/or unscented lubricant composition may be favoured by certain individuals, and may yield lubricating compositions which are less allergenic and/or or irritating to a subject for example having sensitive skin.

In another embodiment, the lubricant composition complies with the Scientific Committee on Consumer Safety (SCCS), SCCS Notes of Guidance for the Testing of Cosmetic Ingredients and Their Safety Evaluation—11^{th} Revision (SCCS/1628/21), as adopted at meeting of 30 to 31 Mar. 2021, the contents of which are incorporated by reference.

In another embodiment the lubricating composition is formulated for vaginal and/or penile use. In another embodiment the lubricating composition is formulated for vaginal use. In yet another embodiment the lubricating composition is formulated for penile use.

In one embodiment the lubricating composition comprises no, or sustainably no, silicone-based lubricant or ingredient. In another embodiment, the lubricating composition comprises no silicone-based lubricant. In yet another embodiment, the lubricating composition comprises no ingredient comprising silicon.

In one embodiment, the lubricating composition comprises about, or less than about: 25.0%, 22.5%, 20.0%, 17.5%, 15.0%, 12.5%, 10.0%, 7.5%, 5.0%, 4.0%, 3.0%, 2.0%, 1.0%, 0.5%, or 0%, of at least one of: parabens (for example one or more of: ethylparaben, butylparaben, isobutylparaben, isopropylparaben, methylparaben and propylparaben), chlorophenesin, phenoxyethanol, glycerin and/or propylene glycol. In another embodiment the lubricating composition comprises no, or substantially no parabens. In another embodiment, the lubricating composition comprises less than 10%, no, or substantially no glycerin. In yet another embodiment, the lubricating composition comprises less than 25%, no or substantially no propylene glycol.

The lubricating composition may be tailored depending on the desired application.

In one embodiment the lubricating composition is formulated so that it can be applied to the skin of an individual. For example by direct application (e.g. from an appropriate container), or by one or more transfers (e.g. from skin-to-skin contact with another individual).

In one embodiment the lubricating composition is formulated as a coating, for example a coating for an article as defined herein.

In one embodiment the lubricating composition is formulated that is suitable for application to an article. For example an article comprising or consisting of: natural rubber latex, or polyisoprene. In one embodiment the article comprises or consists of natural rubber latex. In one embodiment the article comprises or consists of polyisoprene.

pH

Herein the pH range of the lubricating composition may be in a range of about 3.5 to about 5.5, for example in a range of about 3.8 to about 4.5. In one embodiment the pH is about, at least about, or less than about: 3.5, or 3.6, or 3.7, or 3.8, or 3.9, or 4.0, or 4.1, or 4.2, or 4.3, or 4.4, or 4.5, or 4.6, or 4.7, or 4.8, or 4.9, or 5.0, or 5.1, or 5.2, or 5.3, or 5.4, or 5.5.

The pH of the lubricating composition may be modified depending on the application and/or the article to which the lubricating composition is applied.

In some embodiments, the lubricating composition is effective as a vaginal lubricant, wherein the pH may be in a range of about 3.5 to about 5.5, such as about, less than about, or at least about: 3.5, or 3.6, or 3.7, or 3.8, or 3.9, or 4.0, or 4.1, or 4.2, or 4.3, or 4.4, or 4.5, or 4.6, or 4.7, or 4.8, or 4.9, or 5.0, or 5.1, or 5.2, or 5.3, or 5.4, or 5.5.

In some embodiments, the lubricating composition is effective as a penile lubricant, wherein the pH may be in a range of about 3.5 to about 5.5, such as about, less than about, or at least about: 3.5, or 3.6, or 3.7, or 3.8, or 3.9, or 4.0, or 4.1, or 4.2, or 4.3, or 4.4, or 4.5, or 4.6, or 4.7, or 4.8, or 4.9, or 5.0, or 5.1, or 5.2, or 5.3, or 5.4, or 5.5.

### Viscosity

Herein the viscosity of a lubricating composition may be measured according to ASTM D2196-2020.

Herein, the viscosity may be measured in accordance with United States Pharmacopeia (USP) <912>, rotational method using, for example, a Brookfield Viscometer (DV2T LVT), at a testing temperature, for example 25° C.

In one embodiment the lubricating composition is a non-Newtonian fluid.

Herein a lubricating composition may have a viscosity in a range of about 10 to about 10,000 cP, such as in a range of about 1000 to about 5000 cps, or about 2200 to about 3500 cps. For example, a viscosity of about, or at least about: 10 cP, or 50 cP, or 100 cP, or 200 cP, or 300 cP, or 400 cP, or 500 cP, or 600 cP, or 700 cP, or 800 cP, or 900 cP, or 1000 cP, or 1500 cP, or 2000 cP, or 2500 cP, or 3000 cP, or 3500 cP, or 4000 cP, or 4500 cP, or 5000 cP, or 5500 cP, or 6000 cP, or 6500 cP, or 7000 cP, or 7500 cP, or 8000 cP, or 8500 cP, or 9000 cP, or 9500 cP, or 10000 cP

The viscosity of the lubricating composition at 25° C. can be about 1000 cps to about 10,000 cps. For example, a viscosity of about, or at least about: 1000 cP, or 1500 cP, or 2000 cP, or 2500 cP, or 3000 cP, or 3500 cP, or 4000 cP, or 4500 cP, or 5000 cP, or 5500 cP, or 6000 cP, or 6500 cP, or 7000 cP, or 7500 cP, or 8000 cP, or 8500 cP, or 9000 cP, or 9500 cP, or 10000 cP

For a vaginal lubricant, the viscosity of the lubricating composition may be about 1000 cps to less than about 10,000 cps, such as in a range of about 1000 to about 5000 cps, or about 2200 to about 3500 cps. For example, about, at least about, or less than about: 1000 cP, or 1500 cP, or 2000 cP, or 2500 cP, or 3000 cP, or 3500 cP, or 4000 cP, or 4500 cP, or 5000 cP, or 5500 cP, or 6000 cP, or 6500 cP, or 7000 cP, or 7500 cP, or 8000 cP, or 8500 cP, or 9000 cP, or 9500 cP, or 10000 cP.

In one embodiment the lubricating composition has a viscosity, for example a dynamic viscosity, of about, at least about, or less than about: 250 cP, 500 cP, 750 cP, 1000 cP, 1250 cP, 1500 cP, 1750 cP, 2000 cP, 2500 cP, 3000 cP, 3500 cP, 4000 cP, 4500 cP, 5000 cP, 5500 cP, 6000 cP, 6500 cP, 7000 cP, 7500 cP, 8000 cP, 8500 cP, 9000 cP, 9500 cP, or 10000 cP.

### Lubricity

In one embodiment, the lubricating composition is characterised by its lubricity. This can be measured by the coefficient of friction.

In another embodiment the lubricating composition is characterised by its long-lasting lubricity, wherein the composition provides an appropriate lubricity, e.g. in relation to a particular application or use, for an acceptable period of time. For example, a period of time whilst being stored in a container and/or after being exposed to the atmosphere (for example after being removed or dispersed from a container). The “long-lasting” attribute may allow for a longer duration of action for the lubricating composition and/or allow for less frequent applications of the lubricating composition (for example when used as a massage oil or during coitus).

In another embodiment, after being exposed to the atmosphere, applied to an article or body part, the lubricity of the lubricating composition is consistent or substantially consistent after application to a surface of an article or body part after at least: 5 minutes, 10 minutes, 20 minutes, 30 minutes, 40 minutes, 50 minutes, 1 hour, 2 hours, 3 hours, 4, hours or 5 hours.

In another embodiment the lubricating composition is able to provide an appropriate lubricity after being stored, for example for a specific use or application, for a period of about, or at least about: 1 week, 2 weeks, 3 weeks, 4 weeks, 1 month, 2 months, 3 months, 6 months, 12 months, 18 months, 24 months, 30 months, 36 months, 42 months or 48 months. The lubricating composition may be stored at a range of temperatures, including at about, or at least about: 0° C., 15° C. 20° C. 25° C. 30° C. or 40° C. In some embodiments of the lubricating composition can be shelf-stable and will not physically degrade/decompose at room temperature for a period of approximately about, or at least about: 1 month, 2 months, 3 months 4 months, 5 months, 6 months, 7 months, 8 months, 9 months, 10 months, 11 months, 1 year, 1.5 years, 2 years, 2.5 years, or 3 years. In yet another embodiment, the lubricating composition is shelf-stable for at least 3 years. In one embodiment the lubricating composition has sufficient stability for a period of time to resist changes in its physiochemical properties with said period of time. Such changes may include changes over a given time in colour, clarity, and/or odour. In other embodiment, the lubricating composition has sufficient stability over a period of time such that no, or substantially no unacceptable changes (for example with reference to an appropriate standard), occur in relation to parameters such as: pH, viscosity, performance, and/or other measurable product specifications.

In one embodiment the lubricating composition has a long-lasting lubricity of about, or at least about: 100 rounds, 200 rounds, 300 rounds, 400 rounds, 500 rounds, 600 rounds, 700 rounds, 800 rounds, 900 rounds, 1000 rounds, 1100 rounds, 1200 rounds, 1300 rounds, 1400 rounds, 1500 rounds, or 1600 rounds.

In one embodiment the lubricity is measured via a method comprising:
a) Measuring a quantity of a sample of lubricant composition, for example 0.1 g, and spreading the same amount on the back of a hand of a subject. In one embodiment, all the measured amounts are applied on the same hand.
b) Spreading the lubricant composition on the hand and measure the amounts of rounds and time it takes for the lubricant composition to dry up.
c) Once all the lubricant dries off, wash the hand (e.g. with soap) and dry.
d) Repeat the test, for example 2 more times.

### Osmolality

Herein the osmolality of the lubricating composition may be measured in accordance with United States Pharmacopeia (USP) <785>.

In one embodiment the lubricating composition the osmolality of the lubricating composition may be in a range of about 2500 mOsm/kg to about 4500 mOsm/kg, for example in a range of about 3100 to about 4100 mOsm/kg. For example the osmolality may be, be about, be more than about, or less than about: 4500 mOsm/kg, or 4400 mOsm/kg, or 4300 mOsm/kg, or 4200 mOsm/kg, or 4100 mOsm/kg, or 4000 mOsm/kg, or 3900 mOsm/kg, or 3800 mOsm/kg, or 3700 mOsm/kg, or 3600 mOsm/kg, or 3500 mOsm/kg, or 3400 mOsm/kg, or 3300 mOsm/kg, or 3200 mOsm/kg, or 3100 mOsm/kg, or 3000 mOsm/kg, or 2900 mOsm/kg, or 2800 mOsm/kg, or 2700 mOsm/kg, or 2600 mOsm/kg, or 2500 mOsm/kg.

### Microbial Testing

Herein the lubricant composition may be formulated so that it complies with relevant microbial tests. For example, lubricating composition may be measured in accordance with at least one of: United States Pharmacopeia (USP) <51>, <61> and <62>.

In one embodiment the lubricant composition does not allow for the growth of, or reduces the growth of, at least one of: a microbe, a mould and/or a yeast.

In one embodiment, the total aerobic microbial count (TAMC), in accordance with USP <61>, is less than about: 100, or 90, or 80, or 70 or 60, or 50 cfu/g.

In another embodiment, the total combined yeasts/moulds count (TYMC), in accordance with USP <61>, is less than about: 10 cfu/g.

In another embodiment, the antimicrobial effectiveness test (AET), is used to analyse a lubricant composition as defined herein, in accordance with USP <51> for category 2 product, is not less than 2.0 log reduction from the initial count at 14 days, and no increase from the 14 days' count at 28 days for bacteria such as Escherichia coli, Pseudomonas aeruginosa and Staphylococcus aureus and no increase from the initial calculated count at 14 and 28 days for yeasts and moulds such as Aspergillus brasiliensis and Candida albicans.

In yet another embodiment, one or more of the following microorganisms is not present or absence, in accordance with USP <62>, in the lubricating composition: Staphylococcus aureus; Pseudomonas aeruginosa; Escherichia coli; Salmonella spp; Clostridium spp; and/or Candida albicans. In yet another embodiment, none of the following microorganisms are present in the lubricating composition: Staphylococcus aureus; Pseudomonas aeruginosa; Escherichia coli; Salmonella spp; Clostridium spp; and Candida albicans.

### Articles and Uses

Disclosed herein is an article comprising a personal water-based lubricating composition as defined herein.

Also herein is a coating comprising a personal water-based lubricating composition as defined herein.

Also disclosed is use of a lubricating composition as defined herein. In one embodiment the lubricating composition is used as a coating. In another embodiment the lubricating composition is used as a lubricant, wherein the lubricant composition is optionally deposited directly on at least a portion of skin on an individual.

In one embodiment the lubricating composition is formulated and/or used as a massage gel or liquid.

The lubricating composition may be used in one or more intimate care applications, for example, as a personal lubricant involving one or more body parts, for example, private body parts, on which such a composition, is placed and/or used during sexual activity. In one embodiment the lubricant composition is effective as a lubricant and is useful in intimate care applications. In another embodiment, the lubricating composition substantially non-irritating to body parts, for example, private body parts of a human such as, for example at least one of: skin and oral, anal, penile, and vaginal mucosa and the like, particularly during sexual activity.

In one embodiment the lubricating composition is disposed on: part of; substantially all of; or all of, at least one surface of an article. The reference to “at least one surface” may be an external surface and/or internal surface of the article.

The lubricating composition may be disposed onto at least a portion of an article in a process comprising or consisting of one or more of: spraying, dipping, sponging, rolling in the lubricant composition, dosing, or a mixture thereof.

The lubricating composition may be in the form of a coating on at least a portion of the article. For example the lubricating composition may be applied to at least about: 5%, 10%, 15%, 20%, 25%, 30%, 35%, 40%, 45%, 50%, 55%, 60%, 65%, 70%, 75%, 80%, 85%, 90%, or 95%, of the article. Furthermore, the lubricating composition may be applied to at least about: 5%, 10%, 15%, 20%, 25%, 30%, 35%, 40%, 45%, 50%, 55%, 60%, 65%, 70%, 75%, 80%, 85%, 90%, or 95%, of at least one surface of the article. In one embodiment at least a portion of at least one external surface comprises a lubricating composition as defined herein.

The article may be in the form of, or part of, a contraceptive device.

The article may be in the form of, or part of, a sex toy.

In one embodiment the article may be selected from: a condom, cervical cap, coil, contraceptive diaphragm, pessary, intrauterine device (IUD), sex toy, or spermicide.

The article may be in the form of a condom, for example wherein the lubricating composition covers part thereof or substantially covers an entire length of the condom, packaged or non-packaged. In one embodiment a lubricating composition is disposed upon part of, substantially or all, an external surface, or all external surfaces of a condom. The condom may comprise, consist substantially of, or consist of: an elastomeric material (such as a natural latex rubber), a synthetic material (such as polyisoprene material, polymeric blends, or a like polymeric material). In another embodiment the condom comprises or consists of materials selected from, but not limited to: natural rubber latex, or polyisoprene. In one embodiment the condom comprises, consists essentially of, or consists of natural rubber latex. In one embodiment the condom comprises, consists essentially of, or consists of polyisoprene.

Herein, the compatibility of the lubricating composition with condoms can be measured by ASTM D7661-18 Standard Test Method for Determining Compatibility of Personal Lubricants with Natural Rubber Latex Condoms and/or ISO 19671:2018 Additional lubricants for male natural rubber latex condoms-Effect on condom strength.

Herein the term “natural rubber latex” encompasses cured elastomeric material sourced from Hevea brasiliensis (the traditional rubber tree), non-Hevea rubber such as Parthenium argentatum (guayule), sunflower, goldenrod, and the like, as well as genetically modified variations of these or other biological sources. In some embodiments of the invention, condoms comprise pre-vulcanized and post-vulcanized latex compositions.

In one embodiment the article is a condom. In addition to the lubricating composition, the condom may further comprise one or more further components such as but not limited to: a flavouring, a scent, an active ingredient, and/or the like, and mixtures thereof. Examples of active ingredients include one or more of: medicines (such as one or more of an: anti-viral, anti-microbial, and/or anti-fungal), vasodilators (such as niacin, sildenafil citrate, and/or nitroglycerin), male desensitizing agents (such as benzocaine), warming or cooling liquids (such as caffeine or menthol), spermicides (such as nonoxynol-9).

An article may comprise a lubricating composition in an amount of at least about: 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg, per article.

### Methods of Synthesis

Disclosed herein a method of preparing a lubricating composition as defined herein.

In one exemplary non-limiting method, a lubricating composition was synthesised using the one or more of the following steps:
a) Weigh or measure deionised water (DI water) into a first container, optionally under agitation.
b) Add sodium lactate and/or benzoic acid and/or sorbic acid and/or potassium sorbate to DI water and stir for a period of time, for example about 5 minutes.
c) In a second container, pre-disperse flaxseed gum and hydroxyethyl cellulose and/or xanthan gum and/or hyaluronic acid and/or sclerotium gum in 1,3-propanediol. Stir for a period of time, for example about 5 minutes or until the contents are well dispersed.
d) Add the dispersion of the second container to the first container and continue to stir for a period of time, for about 10 minutes.
e) Add okra gel and/or aloe vera extract to the first container and stir for a period of time, for example 5 minutes.

### Example Embodiments

1. A personal water-based lubricating composition comprising at least one of: a part or extract from an okra plant species; and a part or extract from a flaxseed plant species, wherein the extract from the flaxseed plant species is optionally a gum.
2. The composition according to example embodiment 1, wherein the composition comprises at least one part or extract from an okra plant species.
3. The composition according to any one of the preceding example embodiments, wherein the composition comprises at least one part or extract from a flaxseed plant species.
4. The composition according to any one of the preceding example embodiments, wherein the composition comprises at least one part or extract from an okra plant species and a flaxseed plant species.
5. The composition according to any one of the preceding example embodiments, wherein the okra plant species belongs to the genus Abemoschus.
6. The composition according to any one of the preceding example embodiments, wherein the okra plant species is selected from one or more of: Abelmoschus esculentus, Abelmoschus caillei, Abelmoschus manihot, Abelmoschus ficuneus, Abelmoschus moschatus, or a mixture of any of the two or more species thereof.
7. The composition according to any one of the preceding example embodiments, wherein the flaxseed plant species belongs to the genus Linum.
8. The composition according to any one of the preceding example embodiments, wherein the flaxseed plant species is selected from one or more of: Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linurn perenne var. lewisii, Linum pratense and Linum trigynum, or a mixture of any of the two or more species thereof.
9. The composition according to any one of the preceding example embodiments, wherein each extract or part thereof is independently present in an amount of about, or at least about: 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% w/w.
10. The composition according to any one of the preceding example embodiments, wherein the extract or part thereof is provided in the form of: a liquid, an oil, a gel, a powder, a granule, or a mixture thereof.
11. The composition according to any one of the preceding example embodiments, wherein the composition further comprises at least one further component or excipient which is a natural ingredient.
12. The composition according to any one of the preceding example embodiments, wherein the composition further comprises at least one further component or excipient which is a natural ingredient which is an extract from a plant or a part thereof.
13. The composition according to any one of the preceding example embodiments wherein the composition further comprises at least one further component or excipient which is a natural ingredient which is a natural gum, optionally selected from a botanical or plant source, a seaweed source, and/or produced via bacterial fermentation.
14. The composition according to any one of example embodiments 11 to 13, wherein the at least one natural ingredient is provided in the form of: a liquid, an oil, a gel, a powder or a granule.
15. The composition according to any one of example embodiments 11 to 14, wherein the at least one natural ingredient is present in an amount of at least about: 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% w/w, optionally about or at least about: 0.1% w/w.
16. The composition according to any one of the preceding example embodiments, wherein the composition is a non-Newtonian fluid.
17. The composition according to any one of the preceding example embodiments comprising at least one component which is: a humectant, an emollient, a preservative, a thickener, a perfume/fragrance, a pH adjuster, a pH buffer, an emulsifier, a surfactant, a softener, a cooling and/or tingling agent, or a mixture thereof.
18. The composition according to any one of the preceding example embodiments, wherein the composition comprises or consists of about, or at least about: 40, 42, 44, 46, 50, 52, 54, 56, 58, 60, 62, 64, 66, 68, 70, 72, 74, 76, 78, 80, 82, 84, 86, 88, 90, 92, 94, or 96% w/w; or 40, 42, 44, 46, 50, 52, 54, 56, 58, 60, 62, 64, 66, 68, 70, 72, 74, 76, 78, 80, 82, 84, 86, 88, 90, 92, 94, or 96% w/v; or 40, 42, 44, 46, 50, 52, 54, 56, 58, 60, 62, 64, 66, 68, 70, 72, 74, 76, 78, 80, 82, 84, 86, 88, 90, 92, 94, or 96% v/v, of water.
19. The composition according to any one of the preceding example embodiments, wherein the lubricating composition comprises no or substantially no parabens, optionally selected from: ethylparaben, butylparaben, isobutylparaben, isopropylparaben, methylparaben and/or propylparaben.
20. The composition according to any one of the preceding example embodiments, wherein the composition comprises or consists of about, or at least about: 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%, natural origin content, when measured according to ISO16128-2:2017.
21. The composition according to any one of the preceding example embodiments, wherein the composition is clear, substantially clear or translucent.
22. The composition according to any one of the preceding example embodiments, wherein the lubricating composition has one or more of the following characteristics: a pH in the range of: about 3.5 to about 5.5, or about 3.8 to about 4.5; an osmolality in a range of: about 2500 mOsm/kg to about 4500 mOsm/kg, or about 3100 to about 4100 mOsm/kg; and/or a viscosity in a range of: about 1000 to about 5000 cps, or about 2200 to about 3500 cps, optionally when measured at 25° C.
23. The composition according to any one of the preceding example embodiments, wherein the composition is formulated for penile and/or vaginal applications.
24. The composition according to any one of the preceding example embodiments, wherein the composition is formulated for vaginal use.
25. The composition according to any one of the preceding example embodiments, wherein the composition is formulated for application to the skin of an individual.
26. The composition according to any one of the preceding example embodiments, wherein the composition is compatible with an article comprising or consisting of natural rubber latex.
27. The composition according to any one of example embodiments 1 to 25, wherein the composition is compatible with an article comprising or consisting of polyisoprene.
28. The composition according to any one of the preceding example embodiments, wherein the article is a contraception device, optionally a condom.
29. The composition according to any one of the preceding example embodiments, wherein the lubricity of the composition is consistent or substantially consistent after application to a surface of an article or body part after about, or at least about: 10 minutes, 15 minutes, 20 minutes, 25 minutes, 30 minutes, 35 minutes, 40 minutes, 45 minutes, 50 minutes, 55, minutes, 1 hour, 2 hours, 3 hours, 4, hours or 5 hours.
30. The composition according to any one of the preceding example embodiments, wherein the lubricity of the lubricating composition is consistent or substantially consistent with a silicone-based lubricating composition comprising a siloxane and/or a cyclomethicone.
31. A massage gel or liquid comprising the personal water-based lubricating composition according to any one of example embodiments 1 to 30.
32. A coating comprising the personal water-based lubricating composition according to any one of example embodiments 1 to 30.
33. An article comprising the personal water-based lubricating composition according to any one of example embodiments 1 to 30 or the coating of example embodiment 32.
34. The article according to example embodiment 33, wherein the article is a contraceptive device.
35. The article according to example embodiment 33 or example embodiment 34, wherein the article is selected from: a condom, cervical cap, coil, contraceptive diaphragm, pessary, intrauterine device (IUD), sex toy, or a spermicide.
36. The article according to any one of example embodiments 33 to 35, wherein the article is a condom.
37. Use of the personal water-based lubricating composition according to any one of example embodiments 1 to 30 as a massage gel or liquid.
38. Use of the personal water-based lubricating composition according to any one of example embodiments 1 to 30 as a coating.
39. The use according to example embodiment 38, wherein the coating is for an article according to any one of example embodiments 33 to 35.
40. Use of the personal water-based lubricating composition according to any one of example embodiments 1 to 30 as a lubricant, wherein the lubricant composition is optionally deposited directly on to at least a portion of skin on an individual.

### EXAMPLES

The present disclosure will now be described with reference to the following non-limiting examples and with reference to the accompanying Figures (where relevant).

### Example 1—Example Compositions

A series of exemplary compositions were prepared utilising the general approach as described in paragraph and as shown in Table 1. These composition examples arguably yield similar lubricity compared to commercial silicone-based personal lubricants available on the market.

TABLE 1
Exemplary lubricating compositions.

| | Formula/% by weight | | | | | | |
|---|---|---|---|---|---|---|---|
| | Formula | Formula | Formula | Formula | Formula | Formula | Formula |
| Ingredient | A | B | C | E | F | G | H |
| Flaxseed gum | 0.25-0.50 | 0.25 | 0.25-0.50 | 0.10-0.25 | 0.02-0.10 | 0.02-0.10 | 0.02-0.10 |
| Hyaluronic acid | 0.2 | 0.03-0.05 | — | — | — | — | |
| Xanthan gum | — | — | — | — | 0.3-1.0 | — | 0.0-1.0 |
| Hydroxyethyl | — | — | — | — | — | 1 | 0.0-1.0 |
| cellulose | | | | | | | |
| Sclerotium gum | 0.25-0.5 | 0.25 | 0.25-0.50 | 0.25 | — | 0.0-0.1 | |
| Okra powder | 0.25-1.0 | — | — | — | — | — | |
| Okra extract | — | 0.25 | 0.25-2.00 | 1.0-30.0 | 1.0-2.0 | 1.0-2.0 | 1.0-2.0 |
| Aloe vera extract | 0.0-0.5 | — | — | — | — | — | |
| 1,3-Propanediol | 0.0-40 | 25.0 | 25.0 | 25.0 | 25.0 | 25.0 | 25.0 |
| Potassium | 0.1 | 0.1 | 0.1 | — | — | — | |
| sorbate | | | | | | | |
| Sorbic acid | 0.07 | 0.07 | 0.07 | — | — | — | |
| Sodium lactate | — | — | — | 0.4 | 0.4 | 0.4 | 0.4 |
| Benzoic acid | — | — | — | 0.3 | 0.3 | 0.3 | 0.3 |
| DI water | 58.58-98.13 | 74.03-74.05 | 72.33-74.08 | 43.8-72.95 | 71.28-72.90 | 71.1-72.28 | 71.2-72.28 |

Properties

| | | | | | | | |
|---|---|---|---|---|---|---|---|
| Viscosity, cP^{a)} | 128-9448 | 803-1079 | 485.30-1562 | 12.3-668 | 335-2980 | 1376-2456 | 2456-2980 |
| pH | 5.00-5.98 | 5.27-5.37 | 5.19-5.27 | 4.38-4.59 | 4.42-4.58 | 4.22-4.59 | 4.22-4.58 |
| Lubricity, round | 622->1500 | >1500 | >1500 | >1500 | >1500 | >1500 | >1500 |

^{a)}Measured with Brookfield viscometer, LVT #3, speed 30 rpm. 1 minute.

For the compositions of Table 1, the material selection was performed in accordance with ISO16128:2016 Cosmetics-Guidelines on technical definitions and criteria for natural and organic cosmetic ingredients (and both the Natural Index and Natural Origin Index could be calculated according to ISO16128-2:2017. For example two different compositions of Formula H could yield a Natural Origin Contents of 99.53 and 100.

The lubricity properties of a composition of Formula H was tested. The results are shown in Table 2. Additional characteristics of this exemplary formula are also disclosed in Table 2.

The following method was used to test lubricity:
a. Measure 0.1 g of lubricant and spread the same amount on the back of a left hand. Make sure all the measured amount is applied on the hand.
b. Spread the lubricant on the left hand and measure the amounts of rounds and time it takes for the lubricant to dry up.
c. Once all the lubricant dries off, wash the hand with soap and towel dry.
d. Repeat the test for another 2 times.

TABLE 2
Characterisation of a sample of Formula H.

| Test | Specification | |
|---|---|---|
| Appearance | Translucent gel | |
| Colour | Colourless | |
| Odour | Neutral | |
| Residual | None | |
| pH at 25° C. | 4.15 nominal | (3.8-4.5) |
| Viscosity at 25° C., cps (#3, 30 rpm) | 2850 nominal | (2200-3500) |

| | |
|---|---|
| Osmolality, mOsm/kg | 3100-4100 |
| Lubricity (round) | >1500 |

For Comparison Purpose

| | |
|---|---|
| LO^{a)} Plain lubricity (round) | 290 |
| Silicone composition^{b)} lubricity (round) | >1,500 |

^{a)}This comparative composition comprises: sodium carboxymethyl cellulose (3% w/w); glycerine (9% w/w); phenoxytol (0.5% w/w); and DI water (87.5% w/w).
^{b)}This composition comprises: a mixture of a cyclomethicone and a siloxane.

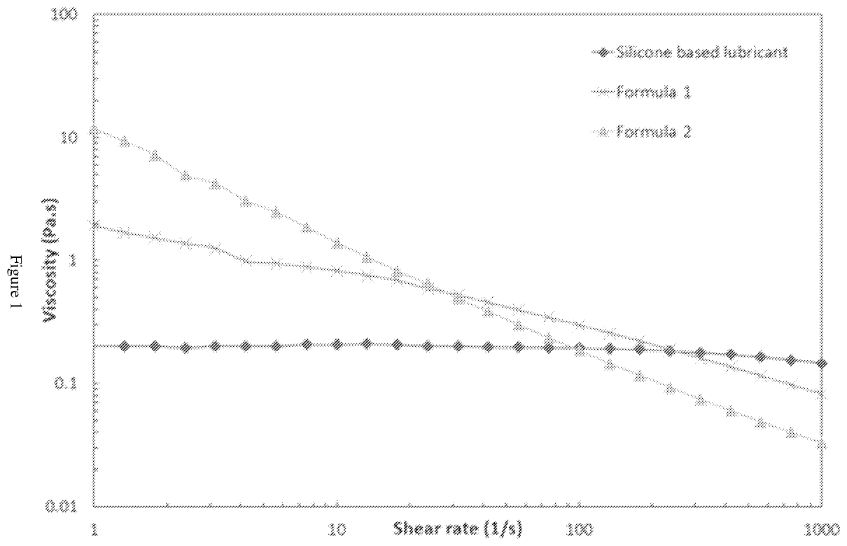

A sample of Formula H was also studied after 50° C. ageing for one month. The results comparing initial: appearance, viscosity and pH are compared to the same characteristics after the ageing. The results are shown in Table 3. Viscosity characterisation of two different variations of Formula H (Formula 1 and Formula 2) are shown in FIG. 1.

TABLE 3
Characterisation of an aged sample of Formula H.

| | | After 1 month |
|---|---|---|
| Age/Characteristic | Initial | at 50° C. |
| Viscosity (cps) | 2160 | 2128 |
| pH | 4.35 | 4.33 |

Additionally, the compositions of Table 1 were also characterised and tested according to USP <51>, <61> and <62>. The above exemplary lubricants met the microbial test per below specifications:
Total aerobic microbial count <100 cfu/g
Total moulds and yeasts <10 cfu/g (measured in accordance with <51> for category 2 products)
An absence of the following microorganisms:
   Staphylcoccus aureus
   Pseudomonas aeruginosa
   Escherichia Coli
   Salmonella sp.
   Clostridium sp.
   Candida albicans

### Example 2—Coefficient of Friction (COF) Testing

Tribology tests of 3 lubricants (silicone gel, Natural Personal Lubricant (NPL), and OFNat), and water were conducted to compare their ability to maintain the COF along 45 minutes of testing (to simulate sexual activity), to review the change in this characteristic over an extended period of time.

The lubricant NPL comprises: xanthan gum, sodium Hyaluronic, glycerin, sodium chloride, sorbic acid, potassium sorbate and water. The lubricant OFNat is within the scope of composition H in Table 1.

### Method

The method used was R&D™10: Determination of coefficient of friction. During the tribology testing, the slope of increased COF is determined, which is calculated from start to end of tribology testing for 45 minutes (maximum sexual intercourse period). 3-balls on a plate geometry move in a circular motion on a silicone gel pad substrate that is lubricated with the lubricant sample. The increasing of COF over the time represents the rate at which lubricity is lost.

### Results

The 4 tested samples (water, NPL, silicone gel and OFNat), were applied on a silicone pad to determine the lubricity for 45 minutes tribology test. All samples except water could maintain the COF at 0.2 approximately for the 45 minutes testing period.

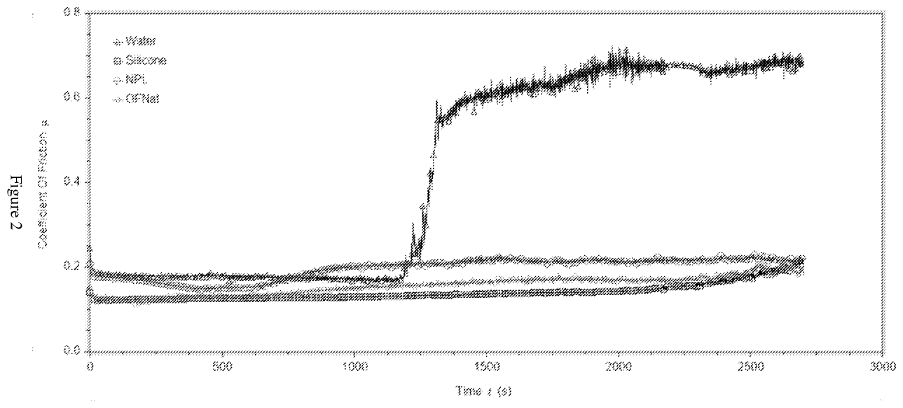

The COF for OFNat was lower than NPL during the testing, indicating a better lubricity. The NPL COF started to fluctuate at around 1500 second meaning that the lubricant started to lose its lubricity, whilst the OFNat COF start to fluctuate at around 2300 second indicating that it can maintain a lubricity for a longer period than NPL. Moreover, the OFNat COF was close to the silicone gel benchmark (see FIG. 2) due to its well-known lubricity.

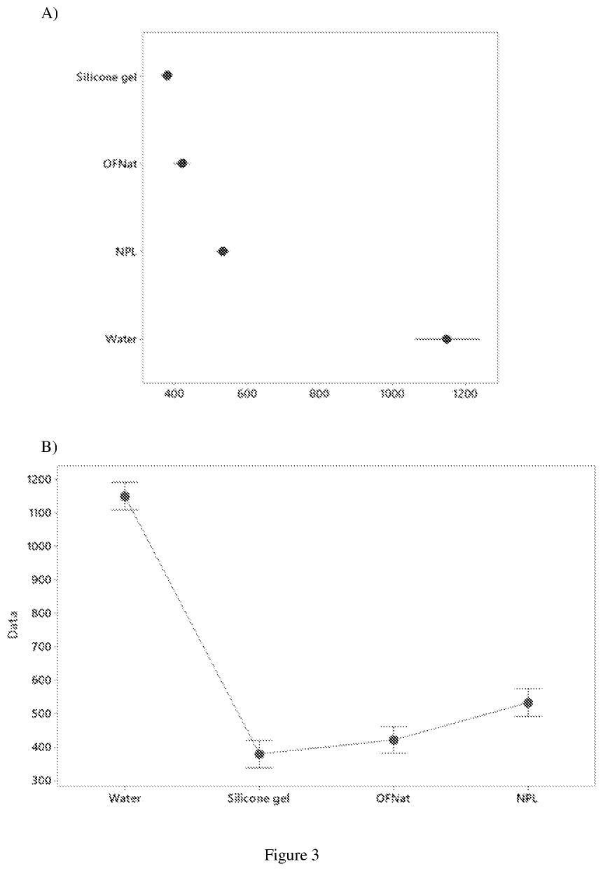

The area under the curve of each tribology test sample was calculated by Rheology Trios software. The results are shown in Table 4, and comparisons are shown FIG. 3, images A) and B). For FIG. 3, image A) equal variances were assumed for the analyses. For FIG. 3, image B), the pooled standard deviation is used to calculate the intervals. In FIG. 3: the null hypothesis is that all means are equal; the alternative hypothesis is that no all means are equal; and the significance level is α=0.05. The analysis of variance is shown in Table 5.

TABLE 4
Area under the curve of all lubricants

| | | Silicone | | |
|---|---|---|---|---|
| Run No. | Water | gel | NPL | OFNat |
| Run 1 | 1228.470 | 378.441 | 556.642 | 427.899 |
| Run 2 | 1279.490 | 380.101 | 550.868 | 381.264 |
| Run 3 | 1283.950 | 375.998 | 560.780 | 426.762 |
| Run 4 | 1303.890 | 381.732 | 505.036 | 417.241 |
| Run 5 | 914.129 | 372.093 | 486.983 | 394.076 |
| Run 6 | 1137.270 | 356.175 | 481.983 | 386.676 |
| Run 7 | 1308.330 | 366.864 | 485.410 | 374.071 |
| Run 8 | 1098.640 | 362.764 | 517.364 | 451.167 |
| Run 9 | 971.782 | 373.320 | 511.724 | 486.676 |
| Run 10 | 1164.570 | 402.161 | 579.989 | 392.522 |
| Run 11 | 1030.120 | 409.454 | 624.511 | 478.927 |
| Run 12 | 1063.370 | 380.398 | 547.688 | 499.391 |
| Run 13 | 1157.600 | 406.147 | 526.215 | 371.617 |
| Mean | 1149.355 | 380.435 | 533.476 | 422.176 |
| Standard | 124.640 | 15.704 | 40.149 | 42.729 |
| Deviation | | | | |

TABLE 5
Analysis of variance for FIG. 3

| Source | DF | Adj SS | Adj MS | F-Value | P-Value |
|---|---|---|---|---|---|
| Factor | 3 | 4994877 | 1664959 | 319.86 | 0.000 |
| Error | 48 | 249852 | 5205 | | |
| Total | 51 | 5244728 | | | |

The overall COF as well as the area under the curve (371.617-499.391) of OFNat lubricant is close to the results for the silicone gel, and significantly lower than the NPL lubricant during the COF testing. As such, it can be concluded that the OFNat lubricant yields better lubricity than NPL lubricant and therefore provides a long lasting lubricating composition.

### Example 3—Stickiness Testing

### Objective

Stickiness testing for 4 lubricants and water was undertaken to compare their retraction force between 2 plates at a temperature of 37° C. to simulate an appropriate environment and emulate a human body.

### Method

The method used was R&D™11: Determination of stickiness. The stickiness of a composition (for example a lubricant) was determined by means of retraction force between 2 plates lubricated with the composition. The higher the retraction force (absolute value), the higher the stickiness.

### Results

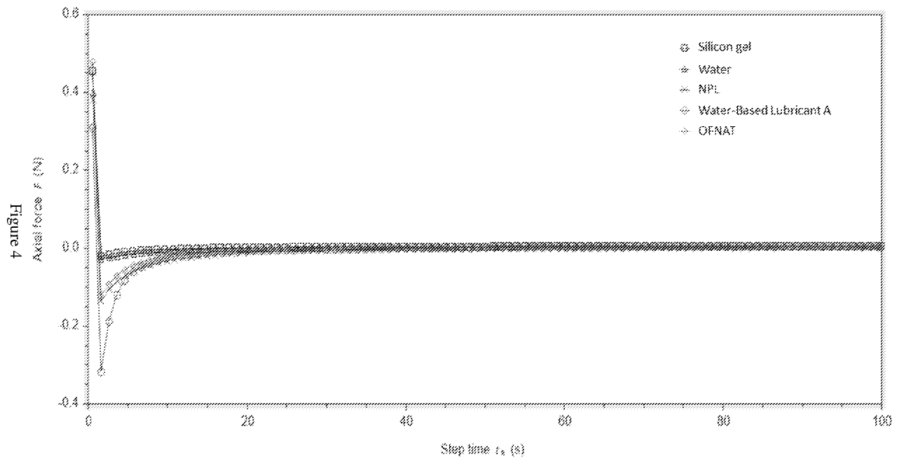

5 different samples (water, water-based lubricant A—as a control, silicone gel, NPL and OFNat) were applied between 2 plates which were then pulled apart to determine the retraction force. All sample show consist results among 13 replicates. An overlay of the average from the 13 replicates for each sample is shown in FIG. 4.

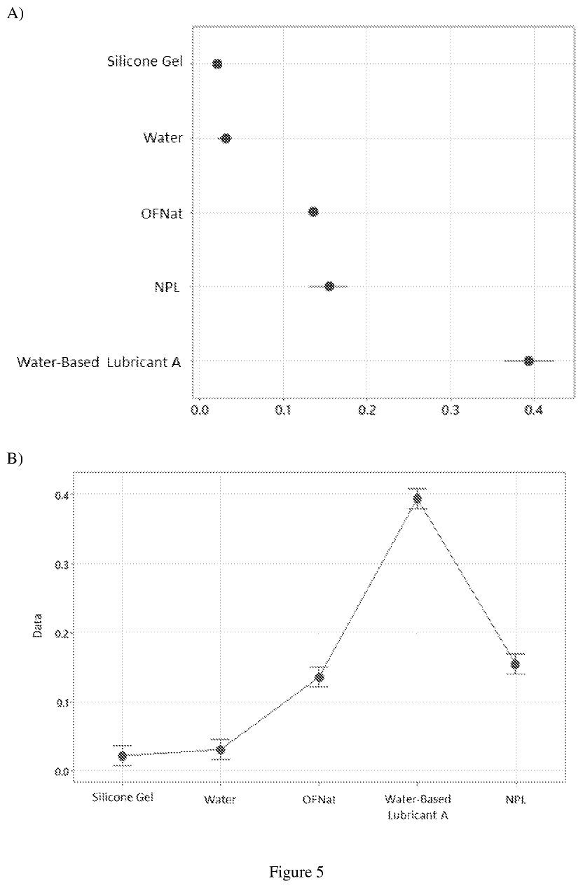

Water-based lubricant A was used as a benchmark and positive control, based on consumer feedback related to stickiness. The results show that water-based lubricant A required the highest force to pull up the upper plate, as can be seen from the high absolute value of axial force at the beginning of retraction step shown in FIG. 4. The silicone gel showed a very low force of retraction as well as water (negative control). The OFNat and NPL water-based lubricant showed similar retraction force values and much lower retraction forces than the water-based lubricant A (Table 6 and FIGS. 4 and 5). In FIG. 5: the null hypothesis is that all means are equal; the alternative hypothesis is that no all means are equal; and the significance level is α=0.05. The analysis of variance is shown in Table 7.

TABLE 6
Maximum axial force (absolute value) of each lubricant

| | Axial Force (N) | | | | |
|---|---|---|---|---|---|
| | | | | Water-based | |
| | | | | Lubricant | |
| | Silicone | | | A | |
| Run No. | Gel | Water | OFNat | (Control) | NPL |
| 1 | 0.019799 | 0.051160 | 0.118289 | 0.319033 | 0.181018 |
| 2 | 0.019300 | 0.047369 | 0.135729 | 0.319379 | 0.197053 |
| 3 | 0.021260 | 0.043617 | 0.127799 | 0.335153 | 0.199241 |
| 4 | 0.021855 | 0.039901 | 0.132389 | 0.451150 | 0.194015 |
| 5 | 0.022453 | 0.037923 | 0.131808 | 0.427455 | 0.186936 |
| 6 | 0.022227 | 0.035360 | 0.127095 | 0.410180 | 0.174117 |
| 7 | 0.021505 | 0.033595 | 0.136418 | 0.403500 | 0.154694 |
| 8 | 0.022425 | 0.029566 | 0.127960 | 0.408363 | 0.149081 |
| 9 | 0.022847 | 0.023500 | 0.130947 | 0.393810 | 0.130608 |
| 10 | 0.022741 | 0.019746 | 0.146230 | 0.398929 | 0.121953 |
| 11 | 0.023781 | 0.015342 | 0.148396 | 0.421560 | 0.117682 |
| 12 | 0.023053 | 0.003359 | 0.150855 | 0.438442 | 0.108952 |
| 13 | 0.023928 | 0.026615 | 0.151686 | 0.394623 | 0.098695 |
| Mean | 0.022090 | 0.031312 | 0.135815 | 0.393967 | 0.154927 |
| Standard | 0.001317 | 0.013005 | 0.010023 | 0.041468 | 0.034769 |
| Deviation | | | | | |

TABLE 7
Analysis of variance for FIG. 5

| Source | DF | Adj SS | Adj MS | F-Value | P-Value |
|---|---|---|---|---|---|
| Factor | 4 | 1.17215 | 0.293037 | | 0.000 |
| Error | 60 | 0.04160 | 0.000693 | | |
| Total | 64 | 1.21374 | | | |

The retraction force of OFNat was lower than the water-based lubricant A and significantly different. It can be concluded that the OFNat water-base lubricant provides a means to avoid issues relating to sticky characteristics, and was found to be less sticky compared to the tested water-based lubricant A.

### Example 4—Fabric Staining Wash Testing

### Objective

To conduct the fabric staining study of OFNat lubricant using an in-house adapted method.

### Method

The method used was R&D001-21-P6. The principal of the test is to perform the fabric staining test of selected fabrics applied, wherein an amount of OFNat lubricant has been applied, and then wash said fabrics. The test results were compared with the same fabric without OFNat lubricant applied (control). In addition, non-water base lubricant (a silicone gel lubricant) was also tested to evaluate ease and effectiveness of washing in comparison to the OFNat lubricant.

### Result:

The results showed that OFNat lubricant do not leave any visible traces on the polyester, cotton, satin and synthetic silk fabric after being hang dried. The washing conditions are recited in Table 8, the results are shown in Table 9. There are silicone gel lubricant staining marks left over on the fabrics (polyester, cotton, satin and synthetic silk) after being washed and dried. These results highlight that the OFNat lubricant does not stain and is easy to wash away from a range of different fabrics.

TABLE 8
Summary of washing conditions for selected compositions and fabrics.

| | Washing conditions | | | | | | Drying Conditions | |
|---|---|---|---|---|---|---|---|---|
| Lubricant | Washing | Water | Wash | Rinse | Spin | Wash/Rinse | Temp./ | Humidity/ |
| Type | Cycle | Level | Time/min. | Time/min. | time/min. | Temp./0° C. | ° C. | % |
| OFNat | Normal | 1 | 17.50 | 1 | 5.25 | 26.1 | 26.1 | 62 |
| Lubricant | (58 min.) | | | | | | | |
| | Delicate | 1 | 12.14 | 2 | 3.26 | 26.1 | 26.1 | 62 |
| | (56 min.) | | | | | | | |
| Silicone Gel | Normal | 1 | 17.50 | 1 | 5.25 | 26.1 | 26.1 | 62 |
| Lubricant | (58 min.) | | | | | | | |
| | Delicate | 1 | 12.14 | 2 | 3.26 | 26.1 | 26.1 | 62 |
| | (56 min.) | | | | | | | |
| Control | Normal | 1 | 17.50 | 1 | 5.25 | 26.1 | 26.1 | 62 |
| (Without | (58 min.) | | | | | | | |
| Lubricant) | Delicate | 1 | 12.14 | 2 | 3.26 | 26.1 | 26.1 | 62 |
| | (56 min.) | | | | | | | |

TABLE 9
Summary of fabric staining results

| | Fabric Type | | | |
|---|---|---|---|---|
| Lubricant | | | | Synthetic |
| Type | Polyester | Cotton | Satin | silk |
| OFNat | No visible | No visible | No visible | No visible |
| Lubricant | spots | spots | spots | spots |
| Silicone Gel | Visible | Visible | Visible | Visible |
| Lubricant | spots | spots | spots | spots |
| Control | No visible | No visible | No visible | No visible |
| (Without | spots | spots | spots | spots |
| Lubricant) | | | | |

### Example 5—Condom Compatibility Testing

### Objective

Testing the condom compatibility of the OFNat lubricant with 3 distinct commercial brands of natural rubber latex (NRL) condoms (A, B, C) and 3 distinct commercial lots of synthetic polyisoprene (PI) condom (D1, D2 and D3) (Table 10).

### Method

The testing was conducted in accordance with ASTM D7661-18.

The crux of the testing was to perform standard strength tests (tensile strength and burst strength tests) on selected condoms treated with OFNat lubricant. These test results were compared to the ones generated on condoms from the same population (control samples), which were not treated with the OFNat lubricant.

TABLE 10
Condoms used for compatibility testing

| Condom | Condom | | Lubricant | Condom |
|---|---|---|---|---|
| Type | Brand | Batch No. | on Condom | Shape |
| Natural | A | 2107442816 | Silicone | Straight |
| Rubber Latex | | | Oil | Shaft |
| Natural | B | T111006 | Silicone | Straight |
| Rubber Latex | | | Oil | Shaft |
| Natural | C | 211018JD | Silicone | Straight |
| Rubber Latex | | | Oil | Shaft |
| Synthetic | D1 | 2109733716 | Silicone | Straight |
| Polyisoprene | | | Oil | Shaft |
| Synthetic | D2 | 2110533716 | Silicone | Straight |
| Polyisoprene | | | Oil | Shaft |
| Synthetic | D3 | 2110573716 | Silicone | Straight |
| Polyisoprene | | | Oil | Shaft |

All samples were tested at the R&D innovation centre, in Surat Thani Thailand.

### Results:

From the results in Table 11, the percent change of airburst volume, airburst pressure, break force and elongation for treated and untreated condoms are within +/−20%. Therefore, the OFNat lubricant is compatible with NRL condom.

From the results in Table 12, the percent change of airburst volume, airburst pressure, break force and elongation for treated and untreated condoms are within +/−20%. Therefore, the OFNat lubricant is compatible with PI SKYN condom.

Therefore, it is concluded that OFNat lubricant is compatible with both natural rubber latex condom and polyisoprene condom.

TABLE 11
Percent change of physical property results for lubricant applied to NRL condom.

| | Condom | | | | | | | | |
|---|---|---|---|---|---|---|---|---|---|
| | A | | | B | | | C | | |
| Negative | | | Positive | | | Positive | | | Positive |
| control vs | Baseline | Treated | Control | Baseline | Treated | Control | Baseline | Treated | Control |
| Airburst | −7.5 | 11.1 | −60.7 | 13.7 | 12.7 | −58.0 | 0.8 | 12.4 | −84.5 |
| volume | | | | | | | | | |
| Airburst | −1.8 | −3.5 | −57.0 | −0.6 | −5.4 | −53.6 | −0.7 | −9.9 | −63.2 |
| pressure | | | | | | | | | |
| Break | −1.0 | 1.2 | −89.9 | −1.4 | −11.0 | −92.1 | −3.4 | −18.9 | −98.4 |
| Force | | | | | | | | | |
| Elongation | 0.0 | 1.8 | −52.7 | −0.2 | −1.4 | −56.4 | −1.0 | −4.4 | −73.0 |
| at break | | | | | | | | | |

TABLE 12
Percent change of physical property results for lubricant applied to PI SKYN condom.

| | Condom | | | | | | | | |
|---|---|---|---|---|---|---|---|---|---|
| | D1 | | | D2 | | | D3 | | |
| Negative | | | Positive | | | Positive | | | Positive |
| control vs | Baseline | Treated | Control | Baseline | Treated | Control | Baseline | Treated | Control |
| Airburst | 0.0 | 1.1 | −71.4 | −2.8 | −7.7 | −78.8 | −0.5 | −3.6 | −76.7 |
| volume | | | | | | | | | |
| Airburst | −1.4 | 2.0 | −57.1 | −1.8 | −10.7 | −57.2 | −3.7 | −2.5 | −56.9 |
| pressure | | | | | | | | | |
| Break | −9.0 | −7.0 | −72.1 | −4.8 | −1.3 | −83.0 | −2.4 | −2.2 | −84.3 |
| Force | | | | | | | | | |
| Elongation | −1.5 | −1.3 | −42.8 | −0.5 | −0.5 | −52.0 | −0.1 | −0.3 | −48.7 |
| at break | | | | | | | | | |

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A personal water-based lubricating composition comprising:
at least a part or extract from an okra plant species; or
at least a part or extract from an okra plant species and a part or extract from a flaxseed plant species.

2. The composition according to claim 1, wherein the composition comprises at least one part or extract from a flaxseed plant species.

3. The composition according to claim 1, wherein:
the okra plant species is selected from the group consisting of: Abelmoschus esculentus, Abelmoschus caillei, Abelmoschus manihot, Abelmoschus ficuneus, Abelmoschus moschatus, and a mixture thereof; and
the flaxseed plant species is selected from the group consisting of: Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linurn perenne var. lewisii, Linum pratense and Linum trigynum, and a mixture thereof.

4. (canceled)

5. (canceled)

6. The composition according to claim 1, wherein the extract or part thereof is present in an amount of at least 0.1% w/w.

7. The composition according to claim 1, wherein the extract or part thereof is provided in the form selected from the group consisting of: a liquid, an oil, a gel, a powder or a granule.

8. The composition according to claim 1, wherein the composition further comprises at least one further component or excipient which is: a natural ingredient; or a natural ingredient which is a natural gum.

9. (canceled)

10. (canceled)

11. The composition according to claim 8, wherein the natural ingredient is:
provided in the form selected from the group consisting of: a liquid, an oil, a gel, a powder, or a granule; or
present in an amount of at least 0.1% w/w.

12. (canceled)

13. The composition according to claim 1, wherein the composition is a non-Newtonian fluid.

14. The composition according to claim 1, further comprising at least one component which is selected from the group consisting of: a humectant, emollient, preservative, a thickener, perfume or fragrance, pH adjuster, pH buffer, emulsifier, surfactant, softener, cooling agent, tingling agent, and a mixture thereof.

15. The composition according to claim 1, wherein the composition comprises or consists of at least 40% w/w water.

16. (canceled)

17. The composition according to claim 1, wherein the composition comprises or consists of at least 95% natural origin content, when measured according to ISO16128-2:2017.

18. (canceled)

19. The composition according to claim 1, wherein the lubricating composition has a pH in the range of 3.5 to 5.5 or 3.8 to 4.5.

20. The composition according to claim 1, wherein the lubricating composition has:
an osmolality in a range of 2500 mOsm/kg to 4500 mOsm/kg, or 3100 to 4100 mOsm/kg; or
a viscosity in a range of: 1000 to 5000 cps, or 2200 to 3500 cps, when measured at 25° C.

21. (canceled)

22. The composition according to claim 1, wherein the composition is formulated for penile or vaginal applications.

23. (canceled)

24. The composition according to claim 1, wherein the composition is formulated for application to the skin of an individual.

25. The composition according to claim 1, wherein the composition is compatible with an article comprising or consisting of a materials selected from the group consisting of: natural rubber latex, or polyisoprene, and a mixture thereof.

26. The composition according to claim 1, wherein the composition is compatible with a contraception device.

27.-35. (canceled)

36. A method of using a personal water-based lubricating composition comprising at least a part or extract from an okra plant species; or at least a part or extract from an okra plant species and a part or extract from a flaxseed plant species, the method comprising: applying the personal water-based lubricating composition as a massage gel or liquid, or as a coating.

37. The method of claim 36, wherein of the personal water-based lubricating composition is formulated as a coating for a contraceptive device.

38.-39. (canceled)

40. The composition according to claim 26, wherein the contraception device is a condom.
